(19) United States
(12) Patent Application Publication
(10) Pub. No.: US 2014/0242540 A1
(43) Pub. Date: Aug. 28, 2014

(54) METHODS OF MAKING AN IMPRESSION OF DENTAL TISSUE AND DENTAL ARTICLES

(71) Applicant: 3M INNOVATIVE PROPERTIES COMPANY, SAINT PAUL, MN (US)

(72) Inventors: Todd D. Jones, Saint Paul, MN (US); Joachim W. Zech, Kaufering (DE); Marc Peuker, Schondorf (DE); Naimul Karim, Maplewood, MN (US)

(73) Assignee: 3M INNOVATIVE PROPERTIES COMPANY, SAINT PAUL, MN (US)

(21) Appl. No.: 14/343,946

(22) PCT Filed: Sep. 18, 2012
(86) PCT No.: PCT/US2012/055894

§ 371 Date: Mar. 10, 2014

Related U.S. Application Data
(60) Provisional application No. 61/543,031, filed on Oct. 4, 2011.

(51) Int. Cl. A61K 6/10; A61C 9/00

(52) U.S. Cl. CPC A61K 6/10; A61C 9/004; A61C 9/0006
USPC 433/71; 433/214

(57) ABSTRACT

Methods of making an impression of dental tissue and dental articles (e.g. suitable for dental impression) are described. In one embodiment, the method comprises providing a hardenable composition, comprising a resin system, a filler system, and an initiator system wherein the hardenable composition is a hardenable self-supporting material having sufficient malleability to be formed into a shape. The method further comprises placing said hardenable composition in contact with dental tissue such that an impression of at least a portion of the dental tissue is formed in the hardenable composition. The method further comprises curing the hardenable composition comprising the impression.

### BACKGROUND

Accurate taking of impressions is a key step in the preparation of a dental crown or bridge. Dental impressions should be an exact replica of the existing dentition, the surrounding tissues and support structures. The exact geometry of the tooth preparation, as well as the anatomy of the surrounding and opposing teeth must be accurately recorded. In addition, the relative position of the maxillary and mandibular arches at rest must be recorded in order to ensure correct alignment of opposing teeth—a process known as bite registration. The dental impressions are used to produce prosthetic or corrective appliances. If distortions or inaccuracies in the dental impression occur, the resultant prosthetic or corrective devices will not fit correctly, causing discomfort, pain, and trauma for the patient.

Traditional impressioning systems use one or more (e.g. two-component) low viscosity, flowable elastomeric materials such as polyvinylsiloxane, polyether, alginate, hydrocolloid, polysulfide, or dental waxes. The CAD/CAM manufacture of prosthetic or corrective appliances in dentistry can be achieved by machining with reference to the optically scanned data. Dental impressions are often subjected to optical scanning by a digital imaging system. The dental articles made with traditional impression materials cannot typically be scanned without the addition of particulate-containing surface treatments.

### SUMMARY

The present invention provides methods of making a dental impression and dental impression articles, using a self-supporting, malleable, curable composition as an impression material. The method and impression article described herein can provide various benefits to the users over the traditional impression materials. Unlike a traditional wax impression, the impressions formed are curable, and thus provide a permanent, non-deformable, and non-brittle impression record. In addition, the impressions are scannable without additional particulates surface treatment.

In one embodiment, a method of making an impression of dental tissue is described. The method comprises providing a hardenable composition, comprising a resin system, a filler system, and an initiator system wherein the hardenable composition is a hardenable self-supporting material having sufficient malleability to be formed into a shape; placing the hardenable composition in contact with dental tissue such that an impression of at least a portion of the dental tissue is formed in the hardenable composition; and curing the hardenable composition comprising the impression.

In other embodiments, dental articles suitable for use as a bite registration impression material are described that are sufficiently malleable to be formed into a shape.

In another embodiment, a hardenable composition is described for use as a dental (e.g. bite registration) impression material, comprising a resin system, a filler system, and an initiator system wherein the hardenable composition is a hardenable self-supporting material having sufficiently malleable to be form a bite registration impression.

### BRIEF DESCRIPTION OF THE DRAWINGS

FIG. 1 is a perspective view of a method of making an impression.

FIG. 2 is a perspective view of an impression comprising a bite registration.

### DETAILED DESCRIPTION

Presently described are methods of making an impression of dental tissue, dental impression articles, and a sufficiently malleable composition for use as an impression material.

In one embodiment, a method of making an impression of dental tissue is described. The method generally comprises providing a hardenable composition. The hardenable composition includes a resin system, a filler system, and an initiator system in the form of a hardenable self-supporting (i.e., free-standing) material with sufficient malleability to be subsequently shaped and then hardened.

The method further comprises placing the hardenable composition in contact with at least a portion of dental tissue and forming an impression of the dental tissue in the hardenable composition. Since the composition is malleable, the dental tissue will imprint or form depressions in the composition corresponding to the shape, location and orientation of the dental tissue. One embodiment, as shown in FIG. 2, is an impression of the dental tissue.

Dental tissue includes the dentition, the surrounding tissues and support structures.

In some embodiments, the step of placing said hardenable composition in contact with dental tissue comprises a subject biting the hardenable composition such that a bite registration impression is formed. A bite registration impression records the shape, location, and orientation of the teeth of the upper jaw in relation to the teeth of the lower jaw when the jaws are in a given bite configuration. This may be obtained by placing any physical form of the hardenable self-supporting malleable material between the occlusional surfaces of opposing arches of a patient or a Typodont (i.e. a model of the oral cavity, including teeth, gingiva, and the palate).

In some embodiments, a complete bite registration impression is formed, i.e. the totality of occlusal surfaces of both arches. Thus, when the patient bites down or Typodont squeezes down on the composition, the bite configuration on both sides of the mouth may be recorded simultaneously.

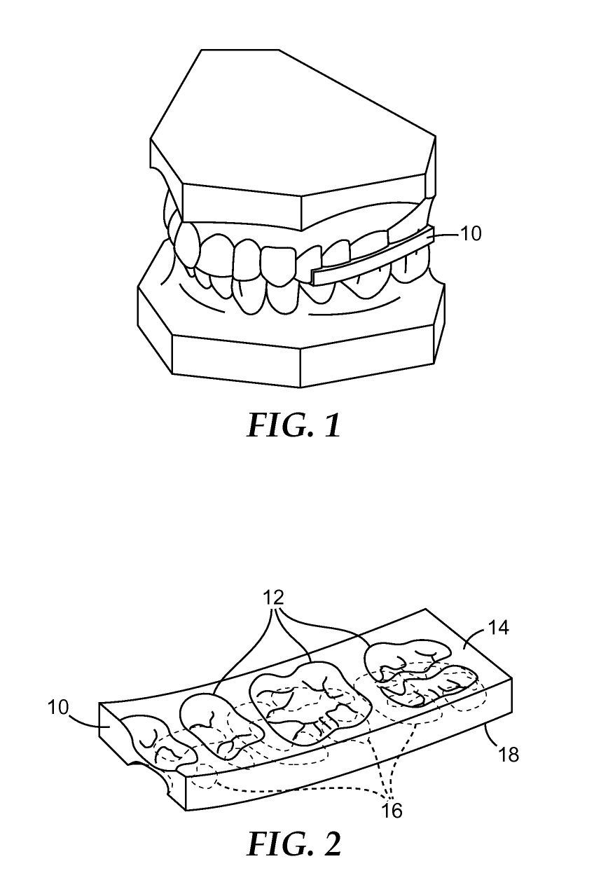

In other embodiments, referring to FIG. 1, a bite registration impression of only a portion of the occlusal surfaces may be formed (e.g. a single tooth, a few teeth, or only the left or right side of the arches), when the patient bites down or Typodont squeezes down on the composition 10.

As shown in FIG. 2, impressions 12 of the teeth and surrounding dental tissues, such as gingiva, in the top jaw may be seen in the top surface 14 of the sufficiently malleable composition 10. Impressions 16 of the teeth and surrounding dental tissues in the bottom jaw may also be seen in the bottom (i.e. opposing) surface 18 of the sufficiently malleable composition 10.

In one embodiment, the bite registration may be comprised of an impression of the surfaces of the anterior teeth. In another embodiment, a bite registration may be comprised of an impression of the surfaces of the posterior teeth.

In the method described herein, the hardenable self-supporting malleable material may be provided in any physical form. In one embodiment, the hardenable composition is an unshaped hardenable composition extruded from a container (e.g., syringe). Alternatively, in some embodiments, the hardenable composition is pre-cut or pre-formed into sheets, films, pads, wafers, or other pre-formed shapes.

Once the desired impression has been achieved, the hardenable composition comprising the impression is then hardened (e.g., cured) by exposing it to heat/radiation to cause activation of the initiator system. This can be done either in a single step, or in multiple steps.

In some embodiments, the hardenable composition can be hardened, typically by exposing it to a dental curing light for a few seconds, if desired, while in the mouth, and then removing it carefully from the mouth and exposing it for final cure to a curing light in a cure chamber, optionally in combination with heat. Alternatively, the hardenable composition can also be sufficiently or completely cured in the mouth by irradiating it with a dental curing light. In another embodiment, the hardenable composition can be cured to a sufficient hardness after being removed from the mouth. One or more of these steps can be carried out in a low or no oxygen inert atmosphere or in vacuum.

In some embodiments, the hardenable composition can be hardened by photocuring.

In some embodiments, the resultant hardened composition has a flexural modulus of at least 100 MPa, and in other embodiments, at least 500 MPa. In some embodiments, the resultant hardened composition has a flexural modulus of at least 700 MPa, and in other embodiments, at least 1000 MPa. In some embodiments, the resultant hardened composition has a flexural modulus of at least 3000 MPa, and in other embodiments, at least 4000 MPa. In some embodiments, the resultant hardened composition has a flexural modulus of at least 5000 MPa, and in other embodiments, at least 6000 MPa.

In the method described herein, the hardened impression can be further scanned, if desired.

The use of dental wax to record the impression has continued to be a popular choice among dentists. However, dental wax has a number of disadvantages for use in dental impression. Because they do not cure, conventional dental waxes can easily break, be deformed, or be damaged when being removed from the model and change their shape under even relatively slight mechanical and above all thermal stress. Therefore, they can be difficult to store as part of the permanent record. Conventional dental waxes are generally heated outside of the patient's mouth for processing and have to then be cooled in the patient's mouth. In this situation, relatively large, uncontrollable volume changes generally occur.

In addition, unlike methods described herein, conventional dental waxes cannot be scanned by a digital imaging system without particulate-containing surface treatment. To achieve a sufficiently accurate scan of dental impressions typically the outer surface of (e.g. two-component) impression material is treated by (e.g. surface treated) particulates (e.g. pigments) of high refractive index in order to facilitate the optical scanning. The pigments mentioned above may improve the scannability of the impression material by brightening and improving contrast. However, the impression made with methods described herein may be scanned or imaged substantially free of a particulate-containing surface treatment, using well known technologies, such as X-rays, three-dimensional X-rays, computer-aided tomographic images or data sets, magnetic resonance images, etc. In an embodiment, the impression can be digitally scanned using a conventional laser scanner to produce the digital data set. The data set produced by the laser scanner system may, of course, be converted to other formats to be integrated into a digital CAD/CAM workflow.

In the method described herein, the hardenable composition includes a resin system, a filler system, and an initiator system in the form of a hardenable self-supporting (i.e., free-standing) material with sufficient malleability to assume the shape of the contacting dental tissue. Such hardenable composition is provided as a one-component composition that does not require mixing before use.

The term “self-supporting” means that the composition is dimensionally stable and will maintain its form (e.g., sheet or film) without significant deformation at room temperature (i.e., about 20° C. to about 25° C.) for at least about two weeks when free-standing (i.e., without the support of packaging or a container). In some embodiments, the compositions are dimensionally stable at room temperature for at least about one month, and in other embodiments, for at least about six months. In some embodiments, the compositions are dimensionally stable at temperatures above room temperature, up to about 40° C., up to about 50° C., or up to about 60° C. This definition applies in the absence of conditions that activate the initiator system and in the absence of an external force other than gravity.

The term “sufficient malleability” means that the self-supporting structure is capable of being shaped and fitted, for example, to a patient's mouth, under a moderate force (i.e., a force that ranges from finger pressure to the amount of force applied when biting).

In many embodiments, the hardenable compositions described herein are “irreversibly hardenable” meaning that after hardening (e.g., curing) the composition loses its malleability, cannot be converted back to a malleable form, and/or cannot be formed into another shape without destroying the external shape of the cured composition.

The combination of highly malleable properties before hardening and high strength (generally, a flexural strength of at least about 25 MPa) after hardening provides an impression material composition with potential advantages.

In some embodiments, the resin system can include one or more monomers, oligomers, and/or polymerizable polymers.

In some embodiments, the resin system includes one or more hardenable organic resins suitable for use in the oral environment, capable of forming a hardened material having sufficient strength.

As used herein, a resin includes one or more monomers, oligomers, and/or polymerizable polymers, including combinations thereof. Although, in this context oligomers and polymers are both used, the terms “polymer” and “polymeric” are used herein to refer to any materials having two or more repeat units, thereby encompassing oligomers. Thus, unless otherwise specified, polymers include oligomers. Furthermore, the term polymer is used herein to encompass both homopolymers and copolymers, and the term copolymer is used herein to encompass materials with two or more different repeat units (e.g., copolymers, terpolymers, tetrapolymers)

In some embodiments, at least some of the resin components include ethylenic unsaturation and are capable of undergoing additional polymerization. In some embodiments, a suitable resin includes at least one ethylenically unsaturated monomer (i.e., includes at least one carbon-carbon double bond).

The (e.g., photopolymerizable) dental compositions may include compounds having free radically reactive functional groups. Examples of useful ethylenically unsaturated compounds include acrylic acid esters, methacrylic acid esters, hydroxy-functional acrylic acid esters, hydroxy-functional methacrylic acid esters, and combinations thereof.

Examples of suitable polymerizable resin components include: mono-, di-, or poly-(meth)acrylates (including acrylates and methacrylates) such as methyl acrylate, methyl methacrylate, ethyl acrylate, isopropyl methacrylate, n-hexyl acrylate, stearyl acrylate, allyl acrylate, glycerol mono- and diacrylate, glycerol triacrylate, ethyleneglycol diacrylate, diethyleneglycol diacrylate, triethyleneglycol dimethacrylate, 1,3-propanediol diacrylate, 1,3-propanediol dimethacrylate, trimethylolpropane triacrylate, 1,2,4-butanetriol trimethacrylate, 1,4-cyclohexanediol diacrylate, pentaerythritol triacrylate, pentaerythritol tetraacrylate, pentaerythritol tetramethacrylate, sorbitol hexacrylate, bis(1-(2-acryloxy))-p-ethoxyphen-yldimethylmethane, bis(1-(3-acryloxy-2-hydroxy))-p-propoxyphenyldimethylme-thane, tris(hydroxyethylisocyanurate)trimethacrylate, 2-hydroxyethyl methacrylate, 2-hydroxypropyl methacrylate, tetrahydrofurfuryl methacrylate, ethylene glycol dimethacrylate, triethylene glycol dimethacrylate, bisGMA, ethoxylated bisphenol A diacrylate, ethoxylated bisphenol A dimethacrylate, biphenyl monomers such as described in US 2011/0171609 and US 2011/0207086, polyethylene glycol dimethacrylate, the bis-acrylates and bis-methacrylates of polyethylene glycols of molecular weight 200-500, copolymerizable mixtures of acrylated monomers such as those of U.S. Pat. No. 4,652,274 (Boettcher et al.), and acrylated oligomers such as those of U.S. Pat. No. 4,642,126 (Zador); unsaturated amides such as (meth)acrylamides (i.e., acrylamides and methacrylamides), methylene bis-acrylamide, methylene bis-methacrylamide, 1,6-hexamethylene bis-acrylamide, diethylene triamine tris-acrylamide, and beta-methacrylamidoethyl methacrylate, diacetone acrylamide, and diacetone methacrylamide; urethane(meth)acrylates; and vinyl compounds such as styrene, diallyl phthalate, divinyl succinate, divinyl adipate, and divinylphthalate. Mixtures of two or more such materials can be used if desired in the resin system.

In some embodiments, the total amount of the resin system is at least about 10 wt-%, at least about 13 wt-%, or at least about 15 wt-%, based on the total weight of the composition. In some embodiments, the total amount of the resin system is no greater than about 60 wt-%, no greater than about 50 wt-%, or no greater than about 40 wt-%, based on the total weight of the composition.

The above-listed components are typically noncrystalline (i.e., amorphous).

In some embodiments, the resin system comprise a crystalline component to impart the (e.g. a noncovalent) three-dimensional structure for maintaining the initial preformed shape such as described in U.S. Pat. No. 7,674,850; incorporated herein by reference. This crystalline component may or may not have a reactive group capable of polymerizing (also including crosslinking). In some embodiments, the crystalline component is polymerizable. In some embodiments, the crystalline component is polymeric (including oligomeric). In some embodiments, the crystalline component is a polymerizable polymeric material.

By crystalline it is meant that the material displays a crystalline melting point at 20° C. or above when measured in the composition by differential scanning calorimetry (DSC). The peak temperature of the observed endotherm is taken as the crystalline melting point. The crystalline component is typically at solid at 20° C.

The crystalline resin component may include at least one material that crystallizes above room temperature (i.e., 20° C. to 25° C.). Such crystallinity, that may be provided by the aggregation of crystallizable moieties present in the component (e.g., when the component is a polymer, in the backbone (i.e., main chain) or pendant substituents (i.e., side chains) of the component), can be determined by well known crystallographic, calorimetric, or dynamic/mechanical methods. This component imparts to the resin system at least one melting temperature (Tₘ) as measured experimentally (for example by DSC) of greater than about 20° C. Typically, this component imparts a Tₘ to the resin system of about 30° C.-100° C. If more than one crystalline material is used in the crystalline component, more than one distinct melting point may be seen.

The average molecular weight of the crystalline component may vary over a broad range. In some embodiments, the molecular weight is less than 10,000 grams per mole (g/mol), and in other embodiments, no greater than about 5000 g/mol. In some embodiments, the molecular weight is at least about 150 g/mol, and in other embodiments, at least about 400 g/mol. At molecular weights less than about 150 g/mol, the crystalline melting point may be too low. At molecular weights greater than about 10,000 g/mol, the crystalline melting point may be too high.

The crystalline monomers suitable for use in the resin system include monomers containing urethane, ether, ester, amide, imide groups, or combinations thereof. In some embodiments, crystalline monomers contain reactive groups capable of polymerizing and/or crosslinking. In some embodiments, crystalline monomers are monomers with a reactive functionality greater than one.

The crystalline polymers (including oligomers) suitable for use in the resin system can have crystalline main chain (i.e., linear) or pendant (i.e., side chain) segments. In some embodiments, materials also contain reactive groups capable of polymerizing and/or crosslinking. In some embodiments, crystalline polymers are crystalline oligomers or prepolymers with a reactive functionality of at least two.

Examples of suitable crystalline materials having crystallizable main chain or backbone segments include, and are not limited to, polyesters (including polycaprolactones), polyethers, polythioethers, polyarylalkylenes, polysilanes, polyamides, polyolefins (preferably, formed from lower, e.g., C₂-C₃, olefins), and polyurethanes.

In some embodiments, crystalline materials are saturated, linear, aliphatic polyester polyols (particularly diols) containing hydroxyl end groups. Examples of commercially available materials useful as the crystalline resin component include some resins available under the trade designation LEXOREZ from Inolex Chemical Co., Philadelphia, Pa. Examples of other polyester polyols are those available as RUCOFLEX from Ruco Polymer Corp., Hicksville, N.Y. Examples of polycaprolactones include those available as TONE 0230, TONE 0240, and TONE 0260 from Dow Chemical Co., Midland, Mich. In some embodiments, crystalline materials are saturated, linear, aliphatic polyester polyols that are modified (e.g., through primary hydroxyl end groups) to introduce polymerizable, unsaturated functional groups, e.g., polycaprolactone diol reacted with 2-isocyanatoethyl methacrylate, methacryloyl chloride, or methacrylic anhydride.

In some embodiments, crystalline polymeric materials include polymeric materials having the following general formula:

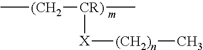

wherein each R is, independently, hydrogen or a (C₁-C₄)alkyl group, X is —CH₂—, —C(O)O—, —O—C(O)—, —C(O)—NH—, —HN—C(O)—, —O—, —NH—, —O—C(O)—NH—, —HN—C(O)—O—, —HN—C(O)—NH—, or —Si(CH₃)₂—, m is the number of repeating units in the polymer, and n is great enough to provide sufficient side chain length and conformation to form polymers containing crystalline domains or regions. In some embodiments, m is at least 2, and in other embodiments, 2 to 100. In some embodiments, n is at least 10. The crystalline polymeric materials may be prepared by the polymerization of monomers containing the pendant (side chain) crystallizable moieties or by the introduction of pendant crystallizable moieties by chemical modification of a polyacrylate, polymethacrylate, polyacrylamide, polymethacrylamide, polyvinyl ester, or poly-alpha-olefin polymers or copolymers. The preparation and morphology/conformational properties that determine the crystalline character of such side chain crystallizable or comb-like polymers are reviewed by Plate and Shibaev, Comb-Like Polymers. Structure and Properties, Journal of Polymer Science, Macromolecular Reviews, 8, 117-253 (1974).

In some embodiments, the polymerizable resin system may comprise a semi-crystalline component as a crystalline component. A semi-crystalline component generally comprises long segments of polymer chains that have regions of both amorphous and crystalline states or phases at 20° C. or above. The amorphous phase is considered to be a randomly tangled mass of polymer chains. A material in a semicrystalline state shows characteristic melting points, above which the crystalline lattices become disordered. The X-ray diffraction pattern of such “semicrystalline” materials generally is distinguished by either concentric rings or a symmetrical array of spots, which are indicative of the nature of the crystalline order.

In some embodiments, the semi-crystalline material is derived from a polyester polymer comprising polycaprolactone repeat units. Polycaprolactone (PCL) homopolymer is a biodegradable polyester with a low melting point of about 60° C. and a glass transition temperature of about −60° C. PCL can be prepared by ring opening polymerization of ε-caprolactone using a catalyst such as stannous octanoate, forming repeat units of polycaprolactone having the general structure:

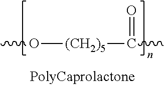

The polycaprolactone polymers utilized herein are typically derived from linear polyester diols derived from caprolactone. One suitable linear polyester diols derived from caprolactone is Capa™ 2125 (Perstorp, Sweden), reported to have a hydroxyl value of 90 mg KOH/g.

In an embodiment, the free-radically polymerizable semi-crystalline resin utilized herein is derived from a polycarpolactone diol having a melt point of 35° C. to 45° C.

The primary hydroxyl groups (i.e. of the diol) are then reacted with a hydroxyl reactive (meth)acrylate compound such as an isocyanatoalkyl(meth)acrylate (e.g. 2-isocyanatoethyl methacrylate (IEM)), methacryloyl chloride, or methacrylic anhydride to convert the hydroxyl group to free-radically polymerizable (e.g. (meth)acrylate) groups.

The free-radically polymerizable semi-crystalline component (e.g. the reaction product of polycaprolactone diol and isocyanatoalkyl(meth)acrylate) has an average molecular weight of no greater than 3,000 g/mole. In some embodiments, the average molecular weight of the free-radically polymerizable semi-crystalline component is no greater than 2700 or 2600 or 2500 g/mole. The molecular weight is typically at least about 400 or 500 g/mol. In some embodiments, the semi-crystalline component has an average molecular weight of at least 1000 g/mole.

The concentration of the (e.g. semi)crystalline component(s) (e.g., polycaprolactone(meth)acrylate resin) in the total dental composition is at least about 0.5 or 1.0 wt-%, based on the total weight of the composition. In some embodiments, the total amount of crystalline component is no greater than about 15 wt-%. In some embodiments, the total amount of crystalline components is no greater than 10 or 8 wt-%. In some embodiments, the concentration of crystalline component ranges from about 0.8 wt-% to about 2.5 wt-%. In other embodiments, the concentration of crystalline component is at least 3.0 or 3.5 wt-%. In some embodiments, the concentration of crystalline component is typically no greater than 6.0, or 5.5, or 5.0 wt-%.

As alternative or in combination with a crystalline component, the polymerizable resin system may comprise an organogelator as described in US 2009/0305196. The organgelator compositions can be flowable, packable, or self-supporting. The term “organogelator” means a low molecular weight compound (generally no greater than 3000 grams per mole) that forms a three-dimensional network structure when dissolved in an organic fluid, thereby immobilizing the organic fluid and forming a non-flowable thermally-reversible gel. In some embodiments the organogelator is a urea-type organogelator, a sugar-based compound, or a combination thereof.

Suitable sugar-based compounds include amino sugar organogelator, dibenzylidene sorbitol, alpha-manno(methyl 4,6-O-benzylidene-alpha-D-mannopyranoside, or a combination thereof.

In some embodiments, the polymerizable resin of the hardenable composition described herein comprises at least one multi-(meth)acrylate aromatic monomer. The selection of components of the polymerizable resin and the concentration of such are generally chosen to minimize polymerization shrinkage.

The polymerization shrinkage can be determined via various methods such as Watts Shrinkage that measure the volumetric change after curing. In some embodiments, low volume shrinkage dental compositions as described herein typically exhibit a Watts Shrinkage of less than 2.5%. In some embodiments, the Watts Shrinkage of the filled dental composition is less than 2.0, or 1.9, or 1.8, or 1.7 or 1.6%.

The hardenable composition described herein typically comprises at least one di-(meth)acrylate aromatic monomer. In some embodiments, the multi-(meth)acrylate monomer is derived from a bisphenol A monomer such as 2,2-bis[4-(2-hydroxy-3-methacryloyloxypropoxy)phenyl]propane (BisGMA). A representative structure for BisGMA is depicted as follows, having a calculated molecular weight of about 512 g/mole:

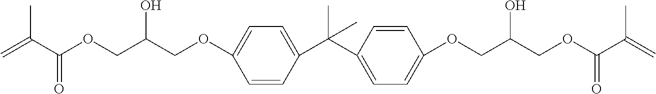 [SMILES: C=C(C)C(=O)OCC(O)COc1ccc(C(C)(C)c2ccc(OCC(O)COC(=O)C(=C)C)cc2)cc1]

In some embodiments, the dental composition comprises a low shrinkage aromatic di(meth)acrylate monomer, such as BisGMA, in combination with one or more other free-radically polymerizable (e.g., methacrylate) monomers. The adjective “other” simply refers to a resin component that is not crystalline and not a low shrinkage monomer. In certain embodiments, the other hardenable components can include diurethane dimethacrylate (UDMA), triethyleneglycol dimethacrylate (TEGDMA), and ethoxylated bisphenol A dimethacrylate as described in U.S. Pat. No. 6,030,606 (Holmes), also referred to herein as “Bis-EMA6”; and 2,2-bis[4-(2-hydroxy-3-methacryloyloxypropoxy)phenyl]propane (Procrylate). In some embodiments, the dental restoration composition comprises BisGMA in combination with ethoxylated bisphenol A dimethacrylate and diurethane dimethacrylate.

The concentration of the other monomers is generally no greater than 20 or 15 wt-% based on the total weight of the composition. In some embodiments, the concentration is less than 15, or 14, or 13 wt-%. In some embodiments, the concentration is typically less than 12, or 11, or 10 wt-%. In other embodiments, the concentration is typically less than 9, or 8, or 7, or 6, or 5 wt-%. The inclusion of 1, or 2, or 3 wt-% of other monomers that are lower in molecular weight than BisGMA may increase the rate of polymerization of the dental article or composition during curing.

The hardenable compositions described herein also comprise a filler.

Fillers for use in the filler system may be selected from a wide variety of conventional fillers for incorporation into resin systems. In some embodiments, the filler system includes one or more conventional materials suitable for incorporation in compositions used for medical applications, for example, fillers currently used in dental restoration compositions. Thus, the filler systems used in the compositions are incorporated into the resin systems, and particularly mixed with the crystalline component of the resin system.

The filler can be an inorganic material. It can also be a crosslinked organic material that is insoluble in the polymerizable resin, and is optionally filled with inorganic filler. The filler is generally non-toxic and suitable for use in the mouth. The filler can be radiopaque, radiolucent, or nonradiopaque. Fillers as used in dental applications are typically ceramic in nature.

Examples of suitable inorganic fillers are naturally occurring or synthetic materials such as quartz, nitrides (e.g., silicon nitride), glasses derived from, for example Ce, Sb, Sn, Zr, Sr, Ba, or Al, colloidal silica, feldspar, borosilicate glass, kaolin, talc, titania, and zinc glass, zirconia-silica fillers; and low Mohs hardness fillers such as those described in U.S. Pat. No. 4,695,251 (Randklev).

Examples of suitable organic filler particles include filled or unfilled pulverized polycarbonates, polyepoxides, and the like. In some embodiments, filler particles are quartz, submicron silica, and non-vitreous microparticles of the type described in U.S. Pat. No. 4,503,169 (Randklev). Mixtures of these fillers can also be used, as well as combination fillers made from organic and inorganic materials.

Optionally, the surface of the filler particles may be treated with a surface treatment, such as a silane-coupling agent, to enhance the bond between the filler and the resin system. The coupling agent may be functionalized with reactive curing groups, such as acrylates, methacrylates, and the like.

The filler particles used to impart a noncovalent structure can be composed of silica, alumina, zirconia, titania, or mixtures of these materials with each other or with carbon. In their synthesized state, these materials are commonly hydrophilic, due to the presence of surface hydroxyl groups. However, the materials may also be modified by treatment with appropriate agents, such as alkyl silanes, in order to modify this character. For example, the surface of a filler particle may be rendered neutral, hydrophobic, or reactive, depending on the desired properties. Fumed silica is a preferred compound for imparting self-supporting character, due to its low cost, commercial availability, and wide range of available surface character.

Fillers may be either particulate or fibrous in nature. Particulate fillers may generally be defined as having a length to width ratio, or aspect ratio, of 20:1 or less, and more commonly 10:1 or less. Fibers can be defined as having aspect ratios greater than 20:1, or more commonly greater than 100:1. The shape of the particles can vary, ranging from spherical to ellipsoidal, or more planar such as flakes or discs. The macroscopic properties can be highly dependent on the shape of the filler particles, in particular the uniformity of the shape.

In some embodiments, particulate filler is finely divided and has an average particle size (preferably, diameter) of less than about 10 micrometers. In some embodiments, micron-size particulate filler has an average particle size of at least about 0.2 microns up to 1 micrometers. Nanoscopic particles have an average primary particle size of less than 200 nm (0.2 microns). The filler can have a unimodal or polymodal (e.g., bimodal) particle size distribution. Micron-size particles are very effective for improving post-cure wear properties. In contrast, nanoscopic fillers are commonly used as viscosity and thixotropy modifiers.

In some embodiments, the filler system comprises nanoscopic fillers. Due to their small size, high surface area, and associated hydrogen bonding, these materials are known to assemble into aggregated networks. Materials of this type (“nanoscopic” materials) have average primary particle sizes (i.e., the largest dimension, e.g., diameter, of unaggregated material) of no greater than about 1000 nanometers (nm). In some embodiments, the nanoscopic particulate material has an average primary particle size of at least about 2 nanometers (nm), and in other embodiments, at least about 7 nm. In some embodiments, the nanoscopic particulate material has an average primary particle size of no greater than about 50 nm, and in other embodiments, no greater than about 20 nm in size. In some embodiments, the average surface area of such filler is at least about 20 square meters per gram (m²/g), and in other embodiments, at least about 50 m²/g, and in other embodiments, at least about 100 m²/g.

In some embodiments, the filler system comprises nanoparticles in the form of nanoclusters, a group of two or more particles associated by relatively weak intermolecular forces that cause the particles to clump together, even when dispersed in a hardenable resin. In some embodiments, nanoclusters can comprise a substantially amorphous cluster of non-heavy (e.g., silica) particles, and amorphous heavy metal oxide (i.e., having an atomic number greater than 28) particles such as zirconia. In some embodiments, the particles of the nanocluster have an average diameter of less than about 100 nm. Suitable nanocluster fillers are described in U.S. Pat. No. 6,730,156 (Mitra et al.).

In some embodiments, the dental composition comprises a (i.e., non-associated) nanoscopic inorganic filler in combination with the (e.g., silica/zirconia) nanoclusters. Such nanoscopic inorganic filler typically comprises silica nanoparticles.

The nano-sized filler may also include fumed silica. In some embodiments, the fumed silica is present in an amount ranging from 1 or 2 up to 5 wt-%. In some embodiments, the fumed silica does not comprise surface modification.

In some embodiments, the total amount of filler system is greater than 50, 60, or 70 wt-%, based on the total weight of the composition. If the filler system includes fibers, the fibers are present in an amount of less than 20 wt-%, based on the total weight of the composition. In some embodiments, the total amount of filler system is no more than about 95 or 80 wt-%, based on the total weight of the composition.

The hardenable compositions also contain an initiator system, i.e., one initiator or a mixture of two or more initiators, which are suitable for hardening (e.g., polymerizing and/or crosslinking) of the resin system, as described in U.S. Pat. Nos. 7,674,850 and 7,816,423. In some embodiments, the initiators are free radical initiators, which may be activated in a variety of ways, e.g., heat and/or radiation. Thus, for example, the initiator system can be a thermal initiator system (e.g., azo compounds and peroxides), or a photoinitiator system. In some embodiments, the initiator system includes one or more photoinitiators. In some embodiments, the initiator system includes at least one photoinitiator active in the spectral region of about 300 nanometers (nm) to about 1200 nm and capable of promoting free radical polymerization and/or crosslinking of ethylenically unsaturated moieties upon exposure to light of suitable wavelength and intensity. A wide variety of such photoinitiators can be used. In some embodiments, the photoinitiator is at least partially soluble in the resin system. In some embodiments, the photoinitiators are sufficiently shelf stable and free of undesirable coloration to permit storage and use under typical dental operatory and laboratory conditions. In some embodiments, the photoinitiators are visible light photoinitiators.

One type of suitable initiator (i.e., initiator system) is described in U.S. Pat. No. 5,545,676 (Palazzotto et al.), which includes a three component or ternary photoinitiator system. This system includes an iodonium salt, e.g., a diaryliodonium salt, which can be a simple salt (e.g., containing an anion such as Cl⁻, Br⁻, I⁻, or C₂H₅SO₃⁻) or a metal complex salt (e.g., containing SbF₅OH⁻ or AsF₆⁻). Mixtures of iodonium salts can be used if desired. The second component in this ternary photoinitiator system is a sensitizer, which is capable of light absorption within the range of wavelengths of about 400 nm to about 1200 nm. The third component in this ternary photoinitiator system is an electron donor and includes amines (including aminoaldehydes and aminosilanes or other amines as described for the first initiator system), amides (including phosphoramides), ethers (including thioethers), ureas (including thioureas), ferrocene, sulfinic acids and their salts, salts of ferrocyanide, ascorbic acid and its salts, dithiocarbamic acid and its salts, salts of xanthates, salts of ethylene diamine tetraacetic acid and salts of tetraphenylboronic acid.

Yet another type of photoinitiator includes acylphosphine oxides, such as those described in EP Application No. 173567 (Ying). In some embodiments, suitable acylphosphine oxides are of the general formula (R⁴)₂—P(═O)—C(═O)—R⁵, wherein each R⁴ is individually a hydrocarbon group, preferably an alkyl group, alicyclic group, aryl group, and aralkyl group, any of which can be substituted with a halo-, alkyl- or alkoxy-group, or the two R⁴ groups can be joined to form a ring along with the phosphorous atom, and wherein R⁵ is a hydrocarbon group, preferably, a S-, O-, or N-containing five- or six-membered heterocyclic group, or a —Z—C(═O)—P(═O)—(R⁴)₂ group, wherein Z represents a divalent hydrocarbon group such as alkylene or phenylene having from 2 to 6 carbon atoms. Examples of suitable acylphosphine oxides include bis(2,4,6-trimethylbenzoyl)phenyl phosphine oxide, for example. Optionally, tertiary amine reducing agents may be used in combination with an acylphosphine oxide. Illustrative tertiary amines include those described above as well as ethyl 4-(N,N-dimethylamino)benzoate and N,N-dimethylaminoethyl methacrylate.

Examples of sensitizers suitable for use in a ternary photoinitiator system include ketones, coumarin dyes (e.g., ketocoumarins), xanthene dyes, acridine dyes, thiazole dyes, thiazine dyes, oxazine dyes, azine dyes, aminoketone dyes, porphyrins, aromatic polycyclic hydrocarbons, p-substituted aminostyryl ketone compounds, aminotriaryl methanes, merocyanines, squarylium dyes, and pyridinium dyes. In some embodiments, sensitizers are ketones (e.g., monoketones or alpha-diketones), ketocoumarins, aminoarylketones, or p-substituted aminostyryl ketone compounds. Examples of particularly preferred visible light sensitizers include camphorquinone, glyoxal, biacetyl, 3,3,6,6-tetramethylcyclohexanedione, 3,3,7,7-tetramethyl-1.2-cycloheptanedione, 3,3,8,8-tetramethyl-1,2-cyclooctanedione, 3,3,18,18-tetramethyl-1,2-cyclooctadecanedione, dipivaloyl, benzil, furil, hydroxybenzil, 2,3-butanedione, 2,3-pentanedione, 2,3-hexanedione, 3,4-hexanedione, 2,3-heptanedione, 3,4-heptanedione, 2,3-octanedione, 4,5-octanedione, and 1,2-cyclohexanedione. In some embodiments, camphorquinone is the sensitizer.

In some embodiments, visible light-induced initiators may be camphorquinone combined with a suitable hydrogen donor (e.g., an amine such as those described above for the first initiator system), and optionally a diaryliodonium simple or metal complex salt, chromophore-substituted halomethyl-s-triazine, or halomethyl oxadiazole. In some embodiments, visible light-induced photoinitiators may be combinations of an alpha-diketone, e.g., camphorquinone with additional hydrogen donors, and optionally a diaryliodonium salt, e.g., diphenyliodonium chloride, bromide, iodide or hexafluorophosphate.

In some embodiments, ultraviolet light-induced polymerization initiators may be ketones, such as benzyl and benzoin, acyloins, and acyloin ethers. In some embodiments, ultraviolet light-induced polymerization initiators may be 2,2-dimethoxy-2-phenylacetophenone available under the trade designation IRGACURE 651 and benzoin methyl ether (2-methoxy-2-phenylacetophenone), both from Ciba Specialty Chemicals Corp., Tarrytown, N.Y.

Various other useful initiators are known in the art, such as described in U.S. Pat. Nos. 7,674,850 and 7,816,423.

The initiator system is present in an amount sufficient to provide the desired rate of hardening (e.g., polymerizing and/or crosslinking). For a photoinitiator, this amount will be dependent in part on the light source, the thickness of the layer to be exposed to radiant energy, and the extinction coefficient of the photoinitiator. In some embodiments, the initiator system is present in a total amount of at least about 0.01, or 0.03, or 0.05 wt-%, based on the weight of the composition. In some embodiments, the initiator system is present in a total amount of no more than about 10, or 5, or 2.5 wt-%, based on the weight of the composition.

The hardenable compositions may contain a surfactant system, i.e., one surfactant or a mixture of two or more surfactants. These surfactants, when used in small amounts may interact with other components of the composition, such as an inorganic filler material, to enhance the formation of a noncovalent three-dimensional structure. Such surfactants can be nonionic, anionic, or cationic. The surfactant(s) can be copolymerizable with the resin system or non-copolymerizable. The desired properties can be achieved when the dental composition is free of surfactant.

In some embodiments, the total amount of surfactant system is at least about 0.05, or 0.1, or 0.2 wt-%, based on the total weight of the composition. In some embodiments, the total amount of surfactant system is no more than about 5.0, or 2.5, or 1.5 wt-%, based on the total weight of the composition.

The hardenable composition may additionally flavorants, medicaments, stabilizers (such as BHT), viscosity modifiers, and the like. Such agents may optionally include reactive functionality so that they will be copolymerized with the resin.

Particularly for the method of the invention, the sufficiently malleable composition compositions can be unshaped (e.g. extruded from a container) or comprise any desirable pre-formed shape. The composition can be molded in a variety of ways including, for example, extruding, injection molding, compression molding, thermoforming, vacuum forming, and pressing.

When the impression material is intended to form an impression of an entire tooth structure, e.g. for the purpose of forming a crown, the impression material typically has a thickness slightly greater than the height of the tooth or teeth. In other embodiments, a dental impression article is described for use in forming a bite registration impression (i.e. only the occulusal surfaces). In this embodiment, the sufficiently malleable material is preferably provided in the form of a planar sheet, having a first major surface, an opposing major surface, and a thickness orthogonal to the major surfaces.

In some embodiments, the thickness of the hardenable composition may be less than 3 mm, and in other embodiments, less than 2 mm. In some embodiments, the thickness of the hardenable composition may be less than 1.9, 1.8, 1.7, 1.6, or 1.5 mm, and in other embodiments, less than 1.4, 1.3, 1.2, 1.1, or 1 mm.

The planar sheet may be molded or cut into various shapes.

In some embodiments, the hardenable composition can be formed into a full dental arch-shaped sheet. Thus, the sheet is generally “U-shaped” and may have a width up to 30 mm. Such arc-shaped sheet may be available in multiple sizes to account for the different sizes of patients' mouths or Typodont models—three sizes is typical in a kit.

In some embodiments, the hardenable composition can be formed into a partial dental arch shape, analogous to a quadrant of an impression tray. Thus, such sheet may be ¼ or ½ of the U-shaped full dental arch-shaped sheet. This embodiment may be also available in multiple sizes, for example, shaped for either one half of the arch, or the anterior region of the arch.

In other embodiments, such as when a bite registration of only one or two opposing teeth is desired, the sheet may be in the form of a disk having a diameter slightly larger than the width of the contacting teeth.

In some embodiments, the hardenable composition can be formed into a strip suitable for impressioning one or more teeth, approximately rectangular in form, sufficient to cover the occlusal surface of those teeth. Dimensions of these strips can be 10-30 mm in width, by 15-100 mm in length.

Any of these preformed shaped articles may be packaged individually in heat sealed pockets in a foil envelope. A multiple of individual packages may be interconnected on a roll which the dentist can remove as individual package from the roll.

In one embodiment, the hardenable composition can be formed into a long strip (up to 30 mm wide by less than 3 mm thick) packaged in a foil envelope and rolled up as a tape. The dental professional can cut off a desired length of this impression composition tape, remove it from the foil envelope, and use as needed.

In some embodiments, the dental impression articles (e.g. suitable for use as a bite registration) impression consist solely of the (e.g. self-supporting) sufficiently malleable hardenable material as described herein.

In other embodiments, the hardenable, self-supporting compositions or articles described herein also comprise a polymeric film in contact with at least a portion ofat least one surface of the hardenable composition or dental articles. In one embodiment, polymeric films are provided on both major surfaces of an (e.g. arch-shaped) sheet of sufficiently malleable material.

In some embodiments, the polymeric film may be used to provide a conformable liner. For certain embodiments, the polymeric films provide a high radial stretch with a relatively low recovery load during elastic recovery. A low recovery load may prevent excessive deformation of the hardenable dental products. For certain embodiments, the polymeric film prevents a hardenable dental material, typically a tacky composite, from being forced through the film and contacting a mold in which the dental product is formed. Preventing appreciable amounts of tacky composite from contacting the mold can also prevent excessive deformation of the hardenable dental product when removed from the mold. Moreover, the polymeric film can protect the product during handling and shipping as well as during application by the practitioner. In some embodiments, the polymeric film may protect the hardenable compositions, materials, or dental articles from conditions that would activate the initiator system and thus cause premature hardening, e.g., such as could result from exposure to light in the case of a photoinitiator. In addition, the polymeric film material optionally conforms to the surfaces of the product, thereby providing additional mechanical strength in order to resist damage during shipping. For example, the hardenable compositions, materials, or dental articles could be packaged in a layer of polyethylene on all sides. The polyethylene provides a mechanical structure and can be sealed to avoid contact with water. If the polyethylene were filled with an appropriate dye, e.g., carbon black, incident light would be absorbed before it could reach the enclosed product. If such a packaging layer is somewhat rigid, and if the packaging film is shaped similar to the dental article described herein, then the packaging could enhance the dimensional stability of the product during shipment and storage.

The liner may be any film which releases from the hardenable dental material. Examples of some suitable materials for the polymeric film may include, but are not limited to, e.g., polypropylenes, polyethylenes, polyurethanes, vinyls, thermoplastic elastomers, elastomeric films (e.g., rubber, latex, etc.), fluorinated polymers (e.g., FEP, PFA, THV, ECTFE, etc.), plasticized PVC, elastic-plastic films (e.g., blends of KRATON and polypropylene), copolymers, water soluble polymers (e.g., selected from the group consisting of polyvinylpyrrolidones, polyvinylpyrrolidone/vinyl acetate copolymers, polyvinyl alcohols, polyethylene oxides, polyacrylamides, polyacrylic acids, polysaccharides and synthetically modified polysaccharides (e.g., cellulose ether polymers), alginates (e.g., sodium alginate), polyethyl oxazolines, esters of polyethylene oxide, esters of polyethylene oxide and polypropylene oxide copolymers, urethanes of polyethylene oxide, urethanes of polyethylene oxide and polypropylene oxide copolymers, etc.).

In some embodiments, the polymeric film may comprise a multi-layer polymeric film, such as described in WO2010/057144. Suitable multi-layer polymeric films comprise at least two dissimilar polymers in separate layers. For example, an outer layer may comprise at least one polymer, and an inner core layer may comprise at least one polymer that is different than at least one polymer comprising the outer layer. For certain embodiments, including any one of the article and method embodiments described herein, the dissimilar polymers differ from each other in a characteristic selected from the group consisting of composition, crystallinity, modulus, maximum elongation, recovery load, surface energy, an optical property, and a combination thereof. Examples of polymers dissimilar in composition may include elastic and plastic polymers, homopolymers and copolymers, polymers of different molecular weights, polymers of different densities, one type of polymer and another type of polymer, for example, polyethylene and sytrene-isoprene-styrene block copolymer, different molecular structure (e.g., linear vs. branched), different amounts of a polymer, different phase morphology, and the like. Crystallinity differences may arise due to differences in comonomer content, differences in branching, differences in molecular weight, and the like. Crystallinity differences may translate to different elongation, modulus, density, and/or recovery properties in the separate layers. Surface energy differences may provide good release from the hardenable dental article and/or from a mold while providing good adhesion between layers. Optical properties include, for example, transparency, opacity, percent haze, surface gloss, color, and the like.

For certain embodiments, the impression article comprises a polymeric liner comprising at least two polymers dissimilar in composition, modulus, and/or recovery load.

For certain embodiments, the multi-layer polymeric liner film has a relatively high visible light transmittance, for example, at least 90 percent transmittance and relatively low haze, for example, less than 10 percent haze.

For certain embodiments, the multi-layer polymeric liner film comprises separate layers have a thickness ratio of 1:2 to 1:100, or 1:3 to 1:50, or 1:5 to 1:20. For certain of these embodiments, the separate layers are an outer layer and a core layer. For certain of these embodiments, these ratios refer to the ratio of an outer layer thickness to a core layer thickness.

For certain of these embodiments, the outer layer has a lower surface energy than the core layer.

For certain of these embodiments, one or more of the polymeric liner films may be removed prior to use of the hardenable dental composite article by the dental practitioner. In certain of these embodiments, the hardenable dental composite article may be used with the liner film in place. Use of the liner film enables control of the adhesion between the composite and the dental tissue being impressioned. The liner film can prevent deformation of the impression, if the impression is removed from the dental tissue prior to cure. Such a liner film must be conformable, in order to not cause distortions of the impression during the impression taking method.

### EXAMPLES

Abbreviations, Descriptions, and Sources of Materials

| | Description and Source of Material (Unless otherwise indicated, |
|---|---|
| Abbreviation | available from Sigma-Aldrich, St. Louis, MO.) |
| BisGMA | 2,2-bis[4-(2-hydroxy-3-methacryloyloxypropoxy)phenyl]propane |
| | (CAS No. 1565-94-2) |
| pbw | Parts by weight |
| Polycaprylactone- | Reaction product of TONE-0230 (Dow Chemical) with IEM as |
| IEM | described in U.S. Pat. No. 7,816,423 |
| TPEG-990 | ethoxylated glycerine having a number average molecular weight of |
| | 990 g/mole (Dow Chemical Company, Midland, MI) |
| Si/Zr filler | zirconia silica filler of average particle size 0.6 to 0.9 micrometers |
| | treated with 3-methacryloxypropyltrimethoxysilane as described in |
| | U.S. Pat. No. 6,030,606 |
| CAB-O-SIL M-5 | untreated amorphous fumed silica (Cabot Corp., Boston, MA) |
| IEM | 2-isocyanatoethyl methacrylate |
| CPQ | camphorquinone |
| TINUVIN P | 2-(2-hydroxy-5-methylphenyl)benzotriazole (available from Ciba- |
| | Geigy Corp, Hawthorne, NY) |
| BHT | 2,6-di-tert-butyl-4-methylphenol |
| EDMAB | ethyl 4-(N,N-dimethylamino)benzoate |
| DPIHFP (DPIPF₆) | diphenyl iodonium hexafluorophosphate (Johnson Matthey, Alpha |
| | Aesar Division, Ward Hill, NJ) |

### Preparation of Malleable Dental Composite

A malleable curable composites (hardenable dental materials) was prepared as described in Examples 1-14 of U.S. Pat. No. 7,816,423. The composite consisted of 10.5 pbw of bisGMA, 9.1 pbw of polycaprolactone-IEM, 0.6 pbw of TPEG-990, 76.7 pbw of Si/Zr filler, 2.2 pbw of CAB-O-SIL M-5, 0.03 pbw of CPQ, 0.2 pbw of EDMAB, 0.1 pbw of DPIHFP, 0.03 pbw of BHT, and 0.3 pbw of Tinuvin P.

### Example 1

Sheets of malleable dental composite were pressed out in a benchtop hydraulic press (Carver Inc., Wabash, Ind.) prewarmed to 60° C. with electrically heated platens. Samples were pressed between 0.05 mm (nominal) thickness sheets of polyester film to a thickness of ˜2 mm using metallic spacers to control the as-formed thickness. The sheets obtained by this process were retained for more than 7 days at room temperature, and were subsequently evaluated by a dentist on a Typodont model (Columbia Dentoform, Long Island City, N.Y.) for their ability to retain a bite registration impression. To carry out this evaluation, the sheets of composite were removed from the polyester film and placed between the opposing arches of the Typodont model. Subsequently, the dentist simulated the squeezing of the malleable dental composite between the opposing arches of the Typodont. The composite was then cured in place to a hard solid using a dental curing light (Elipar S10, 3M-ESPE, Saint Paul, Minn.) from the buccal (closed jaws) and occlusal (open jaws).

The resulting cured impression was not readily deformable. The flexural modulus of the impression material was tested by following the procedure described in International Standard ISO 4049-2009, entitled “Dentistry—Polymer-based filling, restorative and luting materials”. The impression material had a flexural modulus of 7316 (1291) MPa. The resulting impression could be scanned using a laser scanner (Dental Wings 5 Series 3D Laser Scanner, Dental Wings Inc., Montreal, Canada) for integration into a digital CAD/CAM workflow without additional powdering.

### Example 2

Sheets of malleable dental composite were pressed out as described in Example 1 to a thickness of 1 mm or 1.5 mm between sheets of EVA copolymer film (Cotran 9715, 3M Company, St. Paul, Minn.). The resulting sheets were conditioned and evaluated as described in Example 1. In this case, the film was retained on at least one surface (maxillary or mandibular) of the impression material during the impression taking process, and only removed after final cure. Again, a hard composite was obtained after light curing which formed a suitable impression for scanning with a laser scanner without additional powdering.

The complete disclosures of all patents, patent documents, and publications cited herein are expressly incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows and equivalents thereof.

1. A method for making an impression of dental tissue, the method comprising:
providing a hardenable composition, comprising a resin system, a filler system, and an initiator system wherein the hardenable composition is a hardenalbe self-supporting material having sufficient malleablility to be formed into a shape;
placing the hardenable composition in contact with dental tissue such that an impression of at least a portion of the dental tissue is formed in the hardenable composition; and
curing the hardenable composition comprising the impression.

2. (canceled)

3. The method of claim 1 further comprising scanning the dental tissue impression.

4. The method of claim 3, wherein the step of scanning the dental tissue impression comprises scanning the dental tissue impression while substantially free of a particulate containing surface treatment.

5. The method of claim 1, wherein the step of placing said hardenable composition in contact with dental tissue comprises a subject biting the hardenable composition such that a bite registration impression is formed.

6. The method of claim 1, wherein the dental tissue impression is hardened in the mouth.

7. The method of claim 1 wherein the composition is provided as a one-component composition that does not require mixing before use.

8. The method of claim 1, wherein the initiator system comprises free radical initiators.

9. The method of claim 8, wherein the initiator system comprises a photoinitiator system.

10. The method of claim 1, wherein the resin is a polymerizable resin system comprising one or more free-radically polymerizable monomers, oligomers, or polymers.

11. The method of claim 1, wherein the resin system comprises a crystalline resin component, an organogelator, or a mixture thereof.

12. The method of claim 11, wherein the crystalline resin component is a polymeric material having crystallizable pendant moieties and the following general formula: 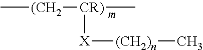
wherein:
R is hydrogen or a (C₁-C₄)alkyl group, X is —CH₂—, —C(O)O—, —O—C(O)—, —C(O)—NH—, —HN—C(O)—, —O—, —NH—, —O—C(O)—NH—, —HN—C(O)—O—, —HN—C(O)—NH—, or —Si(CH₃)₂—;
m is the number of repeating units in the polymer; and
n is great enough to provide sufficient side chain length and conformation to form polymers containing crystalline domains or regions.

13. The method of claim 1, wherein the resin system comprises a free-radically polymerizable semi-crystalline resin component.

14. The method of claim 13, wherein the free-radically polymerizable semi-crystalline resin has a molecular weight no greater than 3000 g/mole.

15. The method of claim 13, wherein the semi-crystalline resin comprises polycaprolactone units.

16. The method of claim 13, wherein the semi-crystalline resin is a reaction product of a polycarprolactone diol and a hydroxyl reactive (meth)acrylate.

17. The method of claim 1, wherein the filler system is present in an amount greater than 60 wt-%, based on the total weight of the composition.

18. The method of claim 1, wherein the filler system is present in an amount greater than 70 wt-%, based on the total weight of the composition.

19. The method of claim 1, wherein the filler system comprises nanoscopic particles.

20. The method of claim 19, wherein the nanoscopic particles comprise fumed silica.

21. The method of claim 1, wherein the hardenable composition is sufficiently malleable such that it can be formed into an impression at a temperature of about 15° C. to 38° C.

22. The method of claim 1, wherein the resin system comprises at least one multi-(meth)acrylate aromatic resin.

23. The method of claim 22, wherein the multi-(meth)acrylate aromatic monomer is a low shrinkage resin derived from bisphenol A.

24. The method of claim 23, wherein the low shrinkage resin is BisGMA

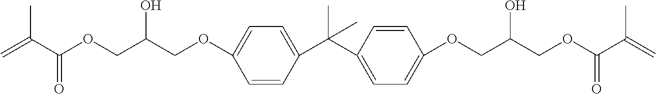 [SMILES: C=C(C)C(=O)OCC(O)COc1ccc(C(C)(C)c2ccc(OCC(O)COC(=O)C(=C)C)cc2)cc1]

25. The method of claim 23, wherein the resin system further comprises other polymerizable resins in addition to the low shrinkage resin in an amount of less than 20 wt-% of the total hardenable composition.

26. (canceled)

27. (canceled)

28. A material comprising a hardenable composition for use as a dental impression material, comprising a resin system, a filler system, and an initiator system wherein the hardenable composition is a hardenable self-supporting material having sufficient malleablility to be form a bite registration impression.

29. A dental article suitable for use as a bite registration impression material, comprising a hardenable composition, wherein the hardenable composition comprises a resin system, a filler system, and an initiator system; and wherein the hardenable composition is a hardenable self-supporting material having sufficient malleablility to be formed into a shape.

30. The material of claim 28, wherein the hardenable composition is in the form of a preformed sheet having a thickness less than 2 mm.

31. The material of claim 28, wherein the hardenable composition is in the form of a preformed sheet having the shape of a full dental arch or a portion thereof.

32. The material of claim 28 further comprising a polymeric film in contact with at least a portion of a surface of the article or material.

33. The article or material of claim 31, wherein the polymeric film is a multi-layer polymeric film comprising at least two dissimilar polymers in separate layers.

34. (canceled)

35. The material of claim 28, wherein the bite registration impression is optically scannable, while also substantially free of a particulate containing surface treatment.
